(19) United States
(12) Patent Application Publication
(10) Pub. No.: US 2003/0008340 A1
(43) Pub. Date: Jan. 9, 2003

(54) Metabolic biosensor and uses thereof

(76) Inventors: John Sheppard, Beaconsfiled (CA); Lucas Vann, Beaconsfield (CA)

Correspondence Address: KLAUBER & JACKSON, 411 HACKENSACK AVENUE, HACKENSACK, NJ, 07601

(21) Appl. No.: 10/165,183

(22) Filed: Jun. 7, 2002

Related U.S. Application Data
(60) Provisional application No. 60/296,160, filed on Jun. 7, 2001.

(51) Int. Cl. C12Q 1/26

(52) U.S. Cl. 435/25

(57) ABSTRACT

The present invention relates to a method and device for analyzing the metabolism of cells involved in a culture or fermentation process. A sample of the culture or fermentation medium is submitted to at least one oxidation-reduction reaction. The device of the invention includes two electrodes that measures the electric conductivity of samples and transmitted a message to an integration electronic system. Thereafter, the difference in the electric conductivity between the untreated and treated samples is indicative of the function of targeted metabolism pathway during the culture of the fermentation process.

### BACKGROUND OF THE INVENTION

Field of the Invention

The invention relates to a method and device for analyzing the metabolism and vitality of cells that are in processes of culture or fermentation.

(b) Description of Prior Art

Different techniques and methods have been used to evaluate culture conditions in a cell culture process. Variables as temperature, pH, and chemical concentrations are measured in different ways for assessing the evolution of conditions in a culture medium.

In a particular case, the carbonic acid generated in the liquid surrounding the cells during a number of metabolic processes partly dissociates into protons and bicarbonate. Together with the nondissociated carbonic acid, the bicarbonate may act as a buffer, reducing the change in pH and preventing distortion of the measured result.

Diacetyl concentration in the brewing process is been important to control, as it is a measure of, beer quality and yeast viability. After completion of the primary fermentation, beer is subjected to a period of maturation to obtain the desired quality. The rate-limiting factor of this maturation period is the reduction of diacetyl concentration and the beer is subjected to high temperatures so that the diacetyl will be degraded by the yeast that metabolize it. If the diacetyl is not degraded during this period, the beer has an undesirable buttery flavor and the yeast must be eliminated and a new culture grown. The essential variables that affect the activities of yeast are time and temperature. The high temperature period, referred to as diacetyl rest, is not good for the yeast but reduces maturation time of the beer from weeks to days and so is economically beneficial. Although there are other methods of diacetyl control the most widely used and effective one is to remove the diacetyl during maturation.

Diacetyl rest is allowed to occur for a fixed time because the initial diacetyl concentration at maturation is not accurately known and must be reduced to a level that will not affect the beer. The yeast is therefore subjected to high temperatures for longer periods than necessary in order to ensure that diacetyl levels are low enough. This is detrimental and “tires out” the yeast, making them less viable.

The quantification of diacetyl in food production in general and in the brewing process in particular is complicated by a number of factors, including its low concentration, the instability of its precursors, its high volatility and the interference of other matrix compounds. Although there are a number of methods currently being employed in diacetyl quantification, including calorimetric, florometric, enzymatic and gas chromatographic (GC) procedures, to date, only the latter when coupled with either an electron-capture (EC) detection device, or a mass spectrometer (MS), has been able to surmount these difficulties and detect diacetyl concentrations below the taste threshold.

GC-EC methodology employs an electron-capture detector that combines a high degree of selectivity with exceptional sensitivity. Its design allows for secondary electrons to be collected, which create a current that can be translated into substrate amounts.

The detector most frequently used in gas chromatography for quantifying diacetyl is the mass spectrometer. GC-MS detection is currently the most comprehensive instrumental analytical technique available in food analysis and represents the most sophisticated technology in the separation and identification of volatile flavor components in many food products. Mass spectrometers are ion-optical instruments that function as a group of subsystems operating on a sample in a sequential order. A sample is vaporized at an inlet producing a beam of gaseous ions. These ions are then separated according to their mass to charge ratio from which the exact mass and abundance of each ion species is determined, Physiological studies on the synthesis of diacetyl and it's precursors along with their kinetics of production and reduction by yeast is made possible with this method.

Due to the limitations of GC-MS and GC-EC technology fermentation practices in the beer industry must rely on qualitative monitoring of yeast performance reducing the possibility of optimal fermentation. Currently there is no method of detecting yeast viability except by the resulting decrease in quality of the final product. Since the rate of diacetyl reduction is a clear indicator of yeast viability, the introduction of biosensor technology will provide a major advantage in overall yeast management that is based on quantitative (not qualitative) on-line monitoring of diacetyl levels.

Presently there is no known device for on-line monitoring of diacetyl levels during beer production.

U.S. Pat. No. 4,424,559 describes modular instrumentation for monitoring and controlling biochemical processes, in particular fermentation processes. The system includes a plurality of function monitoring and control modules each including a microprocessor and associated memory devices, manual input devices and an interface for the receipt of sensor signals and the transmission of control signals. The modules for a plurality of functions have substantially common design and are adapted for relatively quick conversion to another function. The system may include an instrument console adapted to receive a plurality of the function monitoring and control modules as well as incorporating provision for sensor inputs, power inputs, one or more recorders, one or more pumps and/or an interface for an external computer. The back plane of the console is provided with a conductor array interconnecting the various modules, power supply, pumps, recorders, sensor inputs and external computer interface and incorporates provision for the plug-in connection of the respective modules therewith.

U.S. Pat. No. 4,698,224 describes a method for the production of alcoholic beverages by using yeast in high concentration without entailing an increase in the quantity of diacetyls. At least part of the fermentation is conducted under anaerobic conditions to reduce the content of the diacetyls. More specifically, the fermentation is conducted in two zones. In one zone, yeasts are proliferating. In the other zone, yeasts are not proliferating.

U.S. Pat. No. 4,708,875 describes a method for producing fermented alcoholic products that have a low diacetyl content. An acetolactate-converting enzyme is used to decompose acetolactate, which is a precursor of diacetyl. The enzyme is preferably acetolactate decarboxylase contained by Aerobacter aerogenes. The enzyme, in free or immobilized state, may be added during main fermentation or after main fermentation during maturation such as when carrying out malo-lactic fermentation.

U.S. Pat. No. 4,915,959 describes a method for the continuous maturation of fermented beer in which the diacetyl precursors are converted to diacetyl, and the diacetyl is converted to acetoin in order to lower the concentration of diacetyl. The beer is fermented by the use of yeast and after fermentation the yeast is removed and the maturation or lagering of the beer is accomplished by a continuous maturation process which involves heat treating the beer to convert all or substantially all the alpha acetolactate and other diacetyl precursors present to diacetyl, cooling the beer, and feeding the heat treated fermented beer through a reaction column packed with immobilized yeast cells at a flow rate which effects the conversion of the diacetyl to acetoin in order to lower the concentration of the diacetyl to levels which do not result in tastes normally considered unacceptable for a beer.

U.S. Pat. No. 4,978,545 describes a process for the controlled oxygenation of an alcoholic must or wort. A probe, which measures the concentration of dissolved oxygen, is employed. Liquid flow is controlled by signals from the probe. The process comprises putting the must or wort in contact with a side of a membrane permeable to oxygen and putting the other side of this membrane in contact with a gas containing oxygen under partial pressure higher than the partial pressure in oxygen of the liquid. The process is used in wine production plans.

U.S. Pat. No. 5,118,626 describes an apparatus for controlling the fermentation of moromi mash. The apparatus also includes at least one control tank operatively communicating with the storage tank for storing at least one controlling element and supplying the controlling element to the moromi mash in the storage tank, control valves operatively coupled between the control tank and the storage tank for controlling the amount of the controlling element to be supplied to the moromi mash in the storage tank, and a controller for operating the control valves according to analytic results from the automatic multiple analyzer thereby to add the controlling element to the moromi wash in the storage tank to adjust the concentrations of the at least two ingredients of the prescribed amount of moromi mash to target values. The controller periodically actuates a sampling mechanism, and an automatic analyzer adds a controlling element to the moromi mash during the fermentation period.

U.S. Pat. No. 5,306,413 describes an assay apparatus and assay method in which a dehydrogenase in immobilized form and an oxidase in immobilized form are utilized. The invention provides a multiple functional assay apparatus and assay method by which two components, namely oxidized-form substrate, and a reduced-form substrate of a dehydrogenase, can be assayed.

Different techniques in the art are described for achieving more rapid and/or more efficient production of beer, particularly with respect to accelerating the primary fermentation process. For example, it is known that if the temperature during fermentation (either top or bottom fermentation) is increased, the rate of fermentation can be increased and the fermentation time shortened considerably. It is also known that vigorous exogenous agitation (i.e., agitation above that naturally occurring by virtue of the evolution of carbon dioxide by the fermenting yeast) can accelerate the rate of fermentation. However, equally well known is the fact that beers produced according to these methods have an undesirable “winey” off-flavor that has been related to increased amounts of volatile compounds, such as higher alcohols and esters. In addition, these techniques also promote excessive yeast growth.

Another approach to reducing the time required to produce beer is to conduct the operation on a continuous basis. According to different forms of continuous operation, a number of vessels may be employed for the fermentation, each containing a constant volume of wort and yeast in a particular state of fermentation, fresh wort being continuously added at one end of the vessel train and wholly or partly fermented wort being continuously removed from a vessel at the other end of the vessel train. Beers produced according to such methods have not achieved satisfactory flavor, and the process involves complicated equipment and undue risk of contamination as a consequence of the numerous material transfers required and the typically open nature of the vessels.

The very speed with which fermentation is conducted in this continuous process can be self-defeating, a problem that also plagues the earlier-described methods for increasing fermentation rates by means of exogenous agitation and/or increased temperature. Thus, while all these methods may result in an increase in the rate at which sugars in the wort are converted to alcohol, they also limit the amount of time during which yeast, in the process of effecting sugar or carbohydrate conversion, performs other beneficial functions. This is particularly so to the action of yeast on compounds such as diacetyl, which are produced during fermentation. Diacetyl has a distinct buttery flavor that is unacceptable in beers. In conventional fermentation, within the time period in which yeast convert the wort to a desired degree of attenuation, diacetyl is also formed. As a result, the fermented wort can contain undesirably high levels of diacetyl. Further reduction of diacetyl and other compounds such as hydrogen sulfide and acetaldehyde, which are primary components of the “green” aroma of beer after primary fermentation, being accomplished during maturation processes.

Techniques for increasing the speed of fermentation, therefore, limit the time during which the yeast can act upon and absorb diacetyl (and/or precursors of diacetyl) and other compounds. The beer obtained from primary fermentation using these methods has an unacceptably high level of these undesired compounds and must either undergo prolonged maturation to effect reduction of the level of these compounds and/or rely upon other means to effect such reduction. In either case, the beer production is not materially improved over that achieved using conventional fermentation techniques.

It would be highly desirable to be provided with a new method and device allowing monitoring of the metabolism of cells involved in culture and fermentation processes. The monitoring method would allow adjusting the culture or fermentation parameters promptly in processus.

### SUMMARY OF THE INVENTION

One object of the present invention is to provide a method and a device for analyzing the metabolism of cells which avoids the disadvantages of the known devices and which permits determination of condition changes in the culture or fermentation medium during at least one metabolic process of cells in culture, while avoiding physico-chemical changes of the liquid in a manner not beneficial to the cells during the measuring process.

An additional object of the present invention is to enable measurement to be performed very sensitively and very quickly if so desired.

According to the invention, there is provided a device that includes a sensor for measuring electron transfers from an electron donor to an electron acceptor or vice versa in a culture medium containing cultured cells. The electron transfers are measured by light absorbance or by assessing the difference of electric conductivity between an untreated and a treated culture medium sample. The differences in electric conductivity are than correlated with the metabolic state of cells in the culture medium.

Another object of the present invention is to provide a method of monitoring metabolic rate (such as physiological state, cell age, growth rate, or vitality) of cells in a cell culture preparation comprising the steps of:

a) providing a sample of cell culture preparation containing a product to be measured as an indicator of said metabolic rate of said cells;

b) contacting said sample of step a) with a first oxidation or reduction reaction mixture containing an first enzyme and a cofactor, said first enzyme transforming the product to be measured causing reduction or oxidation of the cofactor to obtain a once-reacted sample containing a first transformed product and a reduced or oxidized cofactor;

c) contacting said once-reacted sample of step b) with a second oxidation or reduction reaction mixture containing a second enzyme, said second enzyme transforming the first transformed product of step b) causing reduction or oxidation of the cofactor to obtain a second transformed product and the reduced or oxidized cofactor;

d) comparing the concentration of said reduced or oxidized cofactor in step c) with the concentration of said reduced or oxidized cofactor present in the cell culture preparation, to obtain a difference in concentration; and

e) correlating said difference in concentration of step d) with said metabolic rate.

The cofactor can be for example without limitations selected from the group consisting of pyridine-linked dehydrogenase, flavin-linked dehydrogenase, iron-sulfur protein, a cytochrome, ubiquinone, NAD(H) and NADP(H). The cofactor is more preferably NAD(H) or NADP(H).

The second oxidation or reduction reaction mixture may also comprise a cofactor in which case the cofactor is preferably the same as the one described previously in step b) above.

Determination of the concentration of the reduced or oxidized cofactor in step d) above is preferably determined by measuring light absorbance or by measuring electric conductivity, and correlating said measuring with a measurement of light absorbance or electric conductivity of a known concentration of the cofactor.

In one embodiment of the present invention, the method further comprises before step a) a step of pre-contacting the sample with the second oxidation or reduction reaction mixture of step c) to transform the first transformed product that may be present in the sample.

When there is a pre-contacting step as described above, the comparing step d) is preferably effected between the concentration of the reduced or oxidized cofactor as measured after step c) and the concentration of the reduced or oxidized cofactor as measured before step a) and after the pre-contacting step.

Preferably, the physiological state referred to above is selected from the group consisting of reduction reaction rate, oxidative reaction rate, glycosylation, acetylation, methylation, and carboxylation.

The cells that can be monitored using the present invention are for examples cells selected from the group consisting of microorganism such as yeast or bacteria, animal cell, and plant cell.

The culture preparation can be for example a culture medium, a culture broth, a fermentation medium, or a fermentation broth, such as an alcoholic or a lactic fermentation medium.

In accordance with the present invention, there is also provided a method for the determination of diacetyl concentration as an indicator of cell metabolic rate in a fermentation process, said diacetyl being measured in a sample of a medium obtained from said fermentation process, said method comprising the steps of:

a) contacting said sample with a first oxidation reaction mixture containing a first enzyme for transforming diacetyl into acetoin and an electron acceptor to transform in a first oxidation reaction diacetyl into acetoin producing a reduced electron acceptor;

b) contacting said first oxidation reaction of step a) with a second oxidation reaction mixture containing a second enzyme for transforming acetoin into 2,3-butanediol producing the reduced electron acceptor;

c) comparing the concentration of the reduced electron acceptor of step b) with the concentration of said reduced electron acceptor present in the fermentation process prior to step a); and

d) correlating said difference in concentration of step d) with said diacetyl concentration and said metabolic rate.

In a further embodiment of the present invention, there is also provided a method for monitoring metabolic rate of cells in a cell culture preparation comprising the steps of:

a) providing a sample of cell culture preparation containing a product to be measured as an indicator of said metabolic rate of said cells;

b) contacting said sample of step a) with an oxidation or reduction reaction mixture containing an enzyme and a cofactor, said enzyme transforming the product to be measured causing reduction or oxidation of the cofactor to obtain a reacted sample containing a transformed product and a reduced or oxidized cofactor;

c) comparing the concentration of said reduced or oxidized cofactor in step b) with the concentration of said reduced or oxidized cofactor present in the cell culture preparation, to obtain a difference in concentration; and

d) correlating said difference in concentration of step d) with said metabolic rate.

Still in accordance with the present invention, there is provided a device for measuring a product as an analysis of the metabolism of a cell in a culture medium comprising;

a first reactor comprising a first oxidation or reduction reaction mixture containing an first enzyme and a cofactor, said first enzyme being adapted to transform the product to be measured causing reduction or oxidation of the cofactor;

a second reactor containing a second oxidation or reduction reaction mixture containing a second enzyme, said second enzyme being adapted to transform further the product transformed in the first reactor causing reduction or oxidation of the cofactor;

a detector for determination of the cofactor reduced or oxidized in the first and/or second reactor.

Using the method of the present invention according to which in one embodiment there are two consecutive reduction or oxidation reactions in which the product produced in the first reaction is the substrate for the second reaction. The method thus allows obtaining more sensitive measurements since the oxidized or reduced cofactor produced in both reactions causes an additive effect of the oxidized or reduced cofactor allowing for a more sensitive method.

For the purpose of the present invention the following terms are defined below.

The term “medium” is intended to encompass a broth, a culture medium, a fermentation medium, a fermentation broth, a culture broth, or an incubation medium for cells.

The term spectrophotometry is intended to encompass light absorbancy in the visible or the non-visible spectrum.

### BRIEF DESCRIPTION OF THE DRAWINGS

Having thus generally described the nature of the invention, reference will now be made to the accompanying drawings, showing by way of illustration, and a preferred embodiment thereof, and in which:

FIG. 1 illustrates a biosensor allowing carrying out the method according to one embodiment of the present invention; and

FIG. 2 illustrates a sensing portion of the biosensor.

### DETAILED DESCRIPTION OF THE INVENTION

In accordance with the present invention, there is provided a method of monitoring metabolic reaction rate, the physiological state and/or the vitality of cells in in vitro culture preparation or involved in a fermentation process.

In one embodiment of the invention, there is provided a method to assess, or monitor, the physiological state and/or the viability of cells by measuring a difference in electric conductivity between a non-treated sample of a culture medium or fermentation and a sample of the same culture medium or fermentation broth having been submitted to at least one oxidation-reduction reaction.

The cells may be microorganisms or other living cells, which were taken or in some way derived from a human, animal or plant or other living organisms. An aqueous nutrient solution may be used as a liquid. During investigation of the cell metabolism the cells may change conditions of the culture or fermentation condition by one or more metabolic processes. The changing in culture conditions may be either direct, or the cells may give off substances into the medium, which will then modify the composition of the culture or fermentation medium. In many metabolic processes the cells will produce carbon dioxide, for example, which will then form carbonic acid in the liquid surrounding the cells. Besides, low molecular, aliphatic hydroxy acids, such as lactic acid, may be generated in the cells, which are delivered to the liquid through the cell membrane.

In one embodiment of the present invention, there is provided a device for analyzing the metabolism of cells which avoids the disadvantages of the known devices and which permits the amount of hydrogen transfer in the liquid during at least one metabolic process to be determined while avoiding physico-chemical changes in the culture conditions of the culture medium in a manner not beneficial to the cells during the measuring process. The device of the present invention may as well be embodied in a stick (to be used as a dip-stick) or in a more complex apparatus as will be described hereinafter. Of course, the present description of one embodiment of the present invention is not intended to limit the scope of protection to the specific device described. A person skilled in the art in light of the present description of the device may think of various different embodiment of the present invention, all of which will still have the characteristics or the elements of the device described previously.

In another embodiment of the invention, a sample of a culture or fermentation medium is submitted to a first treatment with a first oxidative or reductive enzyme or product, followed by a second treatment with a second oxidative or reductive enzyme or product. When the first treatment is an oxidation, the second treatment is also an oxidation. On the other side, when the first treatment is a reduction reaction, the second treatment is also a reduction reaction.

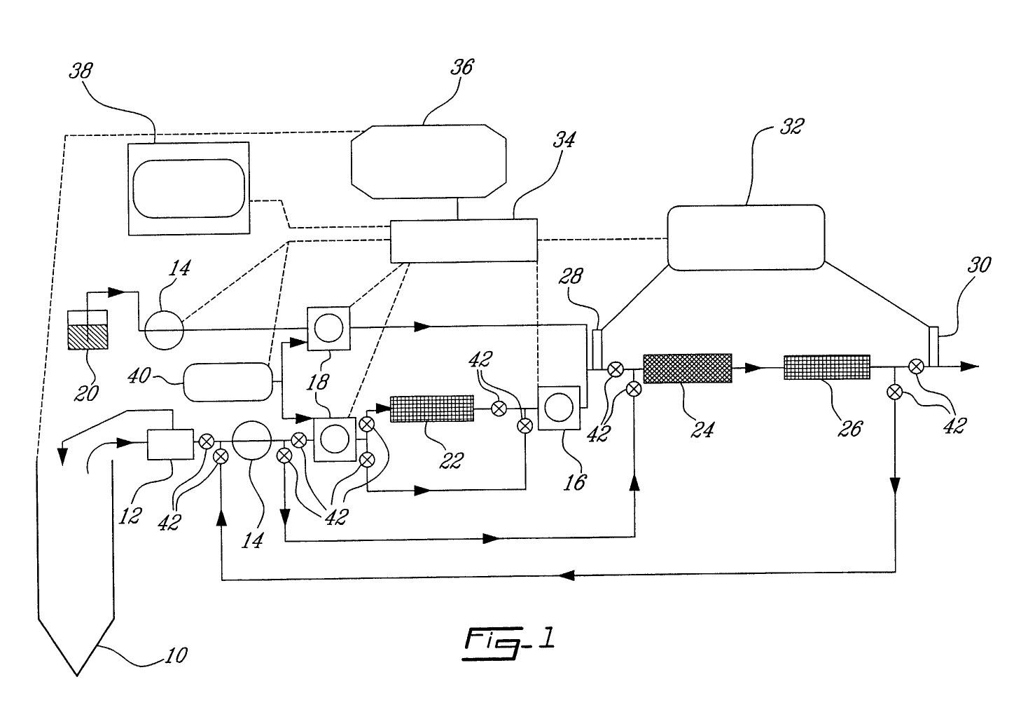

In one embodiment of the invention, there is provided a method that can be performed manually, or through an automated system as illustrated in FIG. 1.

The above enzymes may be used alone or in combination with a hydrogen donor or acceptor. The enzyme, which requires a coenzyme such as, but not limited to, NADH, NADPH, NAD+, or NADP+, may be used also alone. The use of the coenzyme can improve the conversion efficiency, or hydrogen transfer from a donor to an acceptor.

Oxido-reductase for which CH—OH works as a donor may includes alcohol dehydrogenase, alcohol dehydrogenase (used in combination with NADP+ or NAD+ as a coenzyme), butanediol dehydrogenase, acetone dehydrogenase, glycerol dehydrogenase, propanediol phosphate dehydrogenase, and glycerol phosphate dehydrogenase (used in combination with NAD+ as a coenzyme), However, the oxido-reductase shall not be limited to these.

In another embodiment, a buffer solution used in the present invention has a concentration of 0.001 to 1 mol of salt. The salt is preferably a phosphate salt or derivative thereof. Although not specially limited, the buffer solution may include a phosphoric acid buffer solution, a citric acid buffer solution, an acetic acid buffer solution, a tris-hydrochloric acid buffer solution, an ammonium acetate buffer solution, a sodium pyrophosphate buffer solution, a glycine-sodium buffer solution or Good's Buffer. The buffer may also be used for addition of the hydrogen donor or acceptor to the oxidation-reduction reactions.

Diacetyl is also known as 2,3-butanedione having the formula CH3COCOCH3. Acetoin is also known as 3-hydroxy 2-butanone, dimethyketol, or acetyl methylcarbinol having the formula CH3CHOHCOCH3.

Knowing the concentration of diacetyl throughout the maturation process enable the high temperature period to be terminated as soon as possible, benefiting the yeast and reducing overall time for beer production. This may also reduce the need for quality assurance checks to verify that recycled yeast retain their viability and fermentative activity, and as well, reduce the frequency of growing and introducing new yeast cultures which require extra time and capital.

Knowledge of diacetyl concentrations is not only important during the diacetyl rest phase. The evolution of diacetyl in the fermentation process is an indication of the vitality of the yeast culture and the resulting profile of diacetyl levels over time gives an account as to how the yeast are performing at different stages of the brewing process. Diacetyl concentrations peak at a specific time during the fermentation process when the yeast culture is in the optimal physiological state. On line information may provide the profile and peak of the yeast culture being used and its condition may be determined by comparison with optimal yeast culture profiles. The ability to monitor diacetyl levels on line with the method and device of the present invention is important to efficient yeast management, i.e. knowing how to handle the yeast culture in order to keep it in the optimal physiological state. This, in combination with knowing when to end the degradation of diacetyl are two very important factors impacting quality and cost.

According to one embodiment of the present invention, there is provided a method of yeast management that involve the ability to add new yeast cells to the fermentation process at optimum times thereby reducing the reoccurring need to grow new cultures. Yeast cells produce and reduce diacetyl at different rates in relation to their age. Introduction of on-line measurement enables the monitoring and control of yeast age distribution so as to ensure the highest quality product. Determining diacetyl concentrations as fermentation proceeds leads therefore not only to increasing the rate at which the beer is produced, but also the actual quality of the final product and ultimately provide a foundation for the improvements in yeast management.

Still in accordance with the present invention, there is provided a biosensor that is intended to measure diacetyl levels on-line so to provide a profile of diacetyl concentrations during beer production (specifically the fermentation and maturation processes).

On-line monitoring of diacetyl during fermentation may provide an advantage in determining how to effectively treat yeast cultures in order to maintain their optimal physiological state.

On-line measurements throughout the maturation process provides the knowledge of when to terminate the diacetyl rest/degradation period which is not known to date. Optimizing this period is important as it is detrimental to the yeast and also has adverse effects on the beer itself. These are two important factors that not only impact production quality and cost, but also lead to increasing the production rate and can serve as a foundation for future improvements in yeast management in general.

One embodiment of the invention is to allow application of the method of the invention in the brewing industry but it has an anticipated universal application in all alcoholic fermentation processes. The results of implementing this biosensor is of value to the brewing industry specifically and help expand the role biosensors play in introducing new and more effective methods into the food processing industry.

The advantages of the proposed biosensor over this existing technology are numerous. Though GC-MS and GC-EC quantify diacetyl accurately, these are both batch techniques, which require sample preparation, long processing times and expensive equipment. These systems are also complex to handle, requiring the constant supervision of expert technicians. In addition, results arc only obtainable days after taking initial samples. These methods are therefore unable to provide the real time measurements needed to optimize the diacetyl rest period during the brewing process. The proposed biosensor provides a significant advantage in that it provides real time measurements automatically without requiring technical expertise.

Therefore introduction of on-line measurements will enable the brewer to consistently achieve the highest quality product and the shortest possible fermentation times.

A two-part reaction mixture may be conveniently be used to carry out the determination of diacetyl or other metabolic cell markers in accordance with the present invention. In the first part a reaction mixture is utilized containing the bioassay sample, and a solution containing NADPH and a suitable basic buffer solution. The concentration of the NADPH may be in the range from about 0.01 to 1.0 mg/ml. The second part is carried out by reacting the resulting product of the first part in a mixture containing also a desired concentration of NADPH. Any other hydrogen donor depending on the metabolic cell product to be measured in culture medium and test conditions may replace the NADPH.

According to another embodiment of the present invention, the culture conditions and cell metabolic state can be evaluated by measurement of the difference in the electric conductivity of a sample of culture medium or fermentation broth before and after only one oxidation-reduction reaction.

Another embodiment of the invention is to provide a process for producing beer or wine in which the overall time from contacting wort or must with yeast to production of a fermented product of acceptable attenuation and flavor is reduced from the existing in conventional beer and wine-making processes.

Another object of the present invention is to provide a process for producing beer and wine which is capable of utilizing a wide variety of yeast strains, many of which cannot be employed in conventional beer and wine-making processes because, notwithstanding desirable attributes.

Yet another specific embodiment is to provide a process for producing beer or wine which is highly economical in terms of rapid fermentation without development of undesired flavors or aromas, production at the end of a primary fermentation of a beer or wine which does not require an extended maturation period and production of a beer or wine containing low levels of free yeast cells, thereby reducing the burden of subsequent filtration, centrifugation or distillation (for ethanol production).

As noted at the outset, the generalized features of the present invention have applicability to all processes in which it is sought to convert all or a portion of a sugar-containing substrate to ethanol by means of a fermentation process and includes processes for making ethanol per se, processes for making beer and processes for making wine. These features are illustrated hereinafter with reference to beer-making processes. In the course of such illustration, a number of particular features are described which have special applicability to beer-making processes.

During the process of the invention, if conducted in a single fermentation vessel, temperatures can, if desired, be varied throughout the process to attain optimum fermentation and, thereafter, optimum maturation.

According to another embodiment, the method of the present invention enables measurement to be performed very quickly if so desired.

The device may be configured so as to set a starting point of a measurement period and the duration of a measuring and/or integration period with the use of one or more manually operated actuating elements and/or with automatically-operated circuit elements. The circuit elements may be configured so as to integrate the intensity of the electric current flowing through the liquid during the measuring and/or integration period.

FIG. 1 illustrates one embodiment of this invention in which two reactions occur for measuring diacetyl in a brewing fermentation process. The device comprises a fermentor 10, a filter 12, peristaltic pumps 14, injection valve 16, selector valves 18, carrier tank 20, acetoin enzyme pre-reactor 22, a diacetyl enzyme reactor 24, an acetoin enzyme reactor 26, a first electrode 28, a second electrode 30, a detector and recorder 32, an interface 34, a computer 38, a wash solution tank 40 and solenoid valves 42. The use of the device as described herein is exemplified bellow.

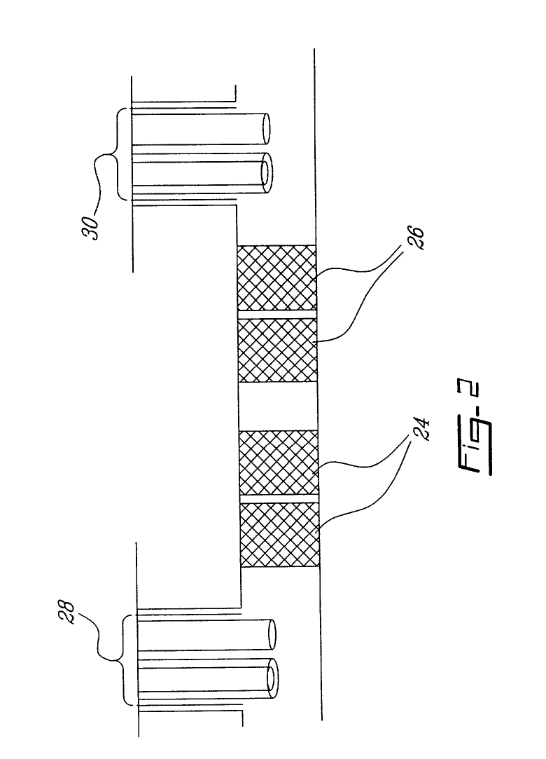

FIG. 2 illustrates the sensing portion of the biosensor, and includes a first electrode 28, a first reactor 24, a second reactor 26 and a second electrode 30.

The present invention will be more readily understood by referring to the following example, which is given to illustrate the invention rather than to limit its scope.

### EXAMPLE I

### Measurement of Metabolites in a Continuous Brewing Process

### Biosensor Design

Due to the low levels of diacetyl that must be measured, the proposed biosensor design incorporates a novel approach in which the product of the first enzyme reaction becomes the substrate for a second reaction thereby increasing the biosensor's sensitivity. An enzyme reactor that must be constructed to meet specific and unique kinetic parameters initiates each reaction. The reactions are as follows; diacetyl is reduced to acetoin by diacetyl reductase and acetoin is then reduced to 2,3-butanediol by butanediol dehydrogenase with NADPH as a cofactor for both reactions. NADPH is reduced and loses a hydrogen in each reaction.

The reduction of NADPH concentration can be measured and converted to a signal that is then proportional to the original concentration of diacetyl based upon the time the sample spends in contact with each enzyme and their respective degradation coefficients.

The design of the proposed biosensor must be such that it will 1) be highly sensitive due to the low levels of diacetyl required to be measured and 2) be integrated so as to not affect the product in any way. The biosensor therefore cannot monitor diacetyl in situ but must be integrated as a flow injection analysis (FIA).

The filter 12 is a plate type cellulose-membrane filter with pore diameter 1-3 microns and maximum flow rate of 0.25-0.7 ml/min. The filter is designed to allow only small molecules like diacetyl to permeate, returning the rest to the fermentor. This decreases interference and fouling of the electrodes. Regular changing of the filter is necessary to prevent rejection of the analyte of interest due to clogged membrane pores.

The peristaltic pump 14 is a multi channel variable peristaltic pump required to transport beer samples at 15-40 □l/min to the injection valve. The pump also transports carrier buffer with NADPH at 15-40 □l/min that is mixed with the sample. 30-80 □I/min of solution then flows through the enzyme reactors for substrate detection.

The injection valve 16 injects samples into the carrier for signal detection by the commercially available Rheodyne inject-ion valve Mod. 7125 (Cotati, Calif., U.S.A.). This valve is equipped with a 50-□l loop to ensure a constant sample flow of 15-40 □l/min is injected into the carrier at the tube depending on the initial diacetyl concentration.

The selector valve 18 acts to switch the sample flow to other solutions that can be externally administered for calibration or wash/purge purposes.

The carrier tank 20 contains 0.1M phosphate buffer pH 7 (the optimal pH for enzyme activity) acts as the carrier. 1.768 E-06 M NADPH is added to the carrier to ensure there is adequate cofactor for the complete reduction of diacetyl to acetoin and then to 2,3 butanediol. Carrier and NADPH solution are pumped through at 15-40 ul/min to mix with the sample.

As there are presently no enzyme reactors for diacetyl and acetoin, the following method of constructing an enzyme reactor will be used as a framework upon which parameters will be optimized for the construction of reactors with the required kinetics.

The acetoin enzyme pre-reactor 22 contains butanediol dehydrogenase enzyme covalently immobilized to commercially available glass beads (Sigma Chemical Co., Canada) with glutaraldehyde. The glass beads are aminopropyl controlled-pore glass (CPG) with a mean pore diameter of 0.07 (go120 mesh). The immobilization procedure is as follows: 0.5 ml of 2.5% gluteraldehyde solution in 0.1M phosphate buffer, pH 7, is added to 0.05 g of aminopropyl-CPG, and the reaction allowed to proceed for 1 hr. The mixture is then filtered and the product washed with distilled water. The glass beads, which now have an active aldehyde group, are added to 1 ml 0.1 M phosphate buffer, pH 7, in which butanediol dehydrogenase enzyme is dissolved. The enzyme and glass mixture is kept at 4° C. for 3 hr and then washed with phosphate buffer to ensure the removal of any unbound enzyme. The glass beads are then packed into Tygon™ tubes to make up the enzyme reactor which has a diameter of 0.01 m and length of 0.482 m. It has been shown that such enzyme reactors can be used for up to two months without any appreciable loss in performance.

The diacetyl enzyme reactor 24 is produced in the same manner as the above acetoin enzyme pre-reactor with the exception that the dimensions are different and butanediol dehydrogenase enzyme is substituted with the diacetyl reductase enzyme. The diameter and length are 0.015 m and 0.4285 m respectively.

The acetoin enzyme reactor 26 is produced in the same manner as the above acetoin enzyme pre-reactor except that the diameter is 0.01 m and the length is 0.4285 m.

First and second electrodes 28 and 30 used for measuring the electric conductivity of medium samples, and therefore as electrochemical sensors for NADPH, are made from spectroscopic graphite rods from Ringsdorff (Bonn, Germany). A 3-mm diameter carbon rod is cut into 2-cm long pieces and placed into a heat shrinkable Teflon™ tube. Electrical contact is made with silver epoxy Eccobond Solder™ from Emerson and Cuming (Milan, Italy). The carbon is then placed in a 7 mm O.D. 6 cm long Teflon™ tube by heat treatment at 300° C. The electrode is then assembled to be ready for NADPH measurements without further treatment according to the procedure previously established (Cagnini A. et al., 1994, Talanta 41:1001-1014). A potential of +500 mV vs. Ag/AgCl is applied to the working electrode in both first and second electrodes. A 6-cm long Ag/AgCl electrode (O,3MKCL) with a diameter of 4-mm O.D. is used as a reference electrode in both instances. The above protocol has been modified from Cagnini et al., (Cagnini A. et al., 1994, Talanta 41:1001-1014).

The detector and recorder 32 consists of a Amel model 559 potentiostat. The current is monitored with an Amel model 868 recorder. Current readings are sent through the interface to the computer for fin-three analysis.

The interface 34 connects the various components of the biosensor and transfers data and/or commands. This component ensures that flow rates meet reactor residence time requirements for adequate NADPH oxidation. As well, it is required to relay commands from the computer to the fermentation control device that alters parameters in the fermentor that affect diacetyl concentrations.

A fermentation control device 36 may be used to control variables in the fermentor that have an effect on diacetyl concentrations. The main variables that would be controlled are time, re-pitching rate and may be temperature, but a number of others could be adjusted as well including pH, dissolved oxygen concentrations, valine levels, and possibly yeast population.

The computer 38 monitors the difference of electric conductivity detected from electrodes 28 to 30 that is related to diacetyl concentrations in the beer sample using a program. The program would also use the information on diacetyl concentrations to adjust parameters in the fermentor through the fermentation control device. In addition there would be feedback to the pump and injection valve to adjust flow rates in order to increase or decrease residence time in the reactors for optimal substrate detection.

A sample of culture or fermentation medium is continuously fed from a fermentor 10 by a feed peristaltic pump 14 through a conduit system to be mixed to a carrier solution containing a hydrogen donor or acceptor, also fed from a carrier tank 20 by a feed peristaltic pump 14, to give a mixed solution. The mixed solution is conducted to a first reactor 24, where occurs a first oxidation-reduction reaction to give an intermediate solution, or a first reacted solution, then the intermediate solution is conducted to a second reactor 26, where a second oxidation-reduction occurs, giving therefore a twice-reacted solution. A first electrode 28 measures the electric conductivity of the mixed solution before entering into the first reactor. A second electrode 30 also measures the electric conductivity of the twice-reacted solution. Several solenoid valves 42 are placed along the system, and for which activation to allow passage of the samples at different stages of the process, is monitored by a fermentation device which themselves is under control of a computer 38.

Another embodiment of the present invention is to provide such a method that can be performed on a brew while the brew is undergoing fermentation processes.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for monitoring metabolic rate of cells in a cell culture preparation comprising the steps of:
a) providing a sample of cell culture preparation containing a product to be measured as an indicator of said metabolic rate of said cells;
b) contacting said sample of step a) with a first oxidation or reduction reaction mixture containing an first enzyme and a cofactor, said first enzyme transforming the product to be measured causing reduction or oxidation of the cofactor to obtain a once-reacted sample containing a first transformed product and a reduced or oxidized cofactor;
c) contacting said once-reacted sample of step b) with a second oxidation or reduction reaction mixture containing a second enzyme, said second enzyme transforming the first transformed product of step b) causing reduction or oxidation of the cofactor to obtain a second transformed product and the reduced or oxidized cofactor;
d) comparing the concentration of said reduced or oxidized cofactor in step c) with the concentration of said reduced or oxidized cofactor present in the cell culture preparation, to obtain a difference in concentration; and
e) correlating said difference in concentration of step d) with said metabolic rate.

2. The method of claim 1, wherein the first enzyme is diacetyl reductase.

3. The method of claim 1, wherein the second enzyme is butanediol dehydrogenase.

4. The method of claim 1, wherein the cofactor is selected from the group consisting of pyridine-linked dehydrogenase, flavin-linked dehydrogenase, iron-sulfur protein, a cytochrome, ubiquinone, NAD(H) and NADP(H).

5. The method of claim 4, wherein the cofactor is NAD(H) or NADP(H).

6. The method of claim 1, wherein the second oxidation or reduction reaction mixture further comprises the cofactor of step b).

7. The method of claim 1, wherein the concentration of the reduced or oxidized cofactor in step d) is determined by measuring light absorbance or electric conductivity, and correlating said measuring with a measurement of light absorbance or electric conductivity of a known concentration of the cofactor.

8. The method of claim 1, further comprising before step a) a step of pre-contacting the sample with the second oxidation or reduction reaction mixture of step c) to transform the first transformed product that may be present in the sample.

9. The method of claim 8, wherein the comparing step is effected between the concentration of the reduced or oxidized cofactor as measured after step c) and the concentration of the reduced or oxidized cofactor as measured before step a) and after the pre-contacting step.

10. The method of claim 1, wherein said metabolic rate is selected from the group consisting physiological state, cell age, growth rate, and vitality.

11. The method of claim 10, wherein said physiological state is selected from the group consisting of reduction reaction rate, oxidative reaction rate, glycosylation, acetylation, methylation, and carboxylation.

12. The method of claim 10, wherein said cells are selected from the group consisting of microorganism, animal cell, and plant cell.

13. The method of claim 12, wherein said microorganism is yeast or bacteria.

14. The method of claim 1, wherein said culture preparation is a culture medium, a culture broth, a fermentation medium, or a fermentation broth.

15. The method of claim 14, wherein said fermentation medium is an alcoholic or a lactic fermentation medium.

16. A method for the determination of diacetyl concentration as an indicator of cell metabolic rate in a fermentation process, said diacetyl being measured in a sample of a medium obtained from said fermentation process, said method comprising the steps of:
a) contacting said sample with a first oxidation reaction mixture containing a first enzyme for transforming diacetyl into acetoin and an electron acceptor to transform in a first oxidation reaction diacetyl into acetoin producing a reduced electron acceptor;
b) contacting said first oxidation reaction of step a) with a second oxidation reaction mixture containing a second enzyme for transforming acetoin into 2,3-butanediol producing the reduced electron acceptor;
c) comparing the concentration of the reduced electron acceptor of step b) with the concentration of said reduced electron acceptor present in the fermentation process prior to step a); and
d) correlating said difference in concentration of step d) with said diacetyl concentration and said metabolic rate.

17. The method of claim 16, wherein said first enzyme is diacetyl reductase.

18. The method of claim 16, wherein said second enzyme is butanediol dehydrogenase.

19. The method of claim 16, wherein the cofactor is selected from the group consisting of pyridine-linked dehydrogenase, flavin-linked dehydrogenase, iron-sulfur protein, a cytochrome, ubiquinone, NAD(H) and NADP(H).

20. The method of claim 19, wherein the cofactor is NAD(H) or NADP(H).

21. The method of claim 16, wherein the second oxidation reaction mixture further comprises the electron acceptor of step a).

22. The method of claim 16, wherein the concentration of the reduced electron acceptor in step c) is determined by measuring light absorbance or electric conductivity, and correlating said measuring with a measurement of light absorbance or electric conductivity of a known concentration of the electron acceptor.

23. The method of claim 16, further comprising before step a) a step of pre-contacting the sample with the second oxidation reaction mixture of step c) to transform acetoin that may be present in the sample.

24. The method of claim 23, wherein the comparing step is effected between the concentration of the reduced electron acceptor measured after step b) and the concentration of the reduced electron acceptor measured before step a) and after the pre-contacting step.

25. The method of claim 16, wherein said electron acceptor is selected from the group consisting of pyridine-linked dehydrogenase, flavin-linked dehydrogenase, iron-sulfur protein, a cytochrome, ubiquinone, NAD(H) and NADP(H).

26. The method of claim 16, wherein said cell is a yeast or a bacterium.

27. A method for monitoring metabolic rate of cells in a cell culture preparation comprising the steps of:
a) providing a sample of cell culture preparation containing a product to be measured as an indicator of said metabolic rate of said cells;
b) contacting said sample of step a) with an oxidation or reduction reaction mixture containing an enzyme and a cofactor, said enzyme transforming the product to be measured causing reduction or oxidation of the cofactor to obtain a reacted sample containing a transformed product and a reduced or oxidized cofactor;
c) comparing the concentration of said reduced or oxidized cofactor in step b) with the concentration of said reduced or oxidized cofactor present in the cell culture preparation, to obtain a difference in concentration; and
d) correlating said difference in concentration of step d) with said metabolic rate.

28. The method of claim 27, wherein the enzyme is diacetyl reductase.

29. The method of claim 27, wherein the cofactor is selected from the group consisting of pyridine-linked dehydrogenase, flavin-linked dehydrogenase, iron-sulfur protein, a cytochrome, ubiquinone, NAD(H) and NADP(H).

30. The method of claim 29, wherein the cofactor is NAD(H) or NADP(H).

31. The method of claim 27, wherein the concentration of the reduced or oxidized cofactor in step c) is determined by measuring light absorbance or electric conductivity, and correlating said measuring with a measurement of light absorbance or electric conductivity of a known concentration of the cofactor.

32. The method of claim 27, wherein said metabolic rate is selected from the group consisting physiological state, cell age, growth rate, and vitality.

33. The method of claim 32, wherein said physiological state is selected from the group consisting of reduction reaction rate, oxidative reaction rate, glycosylation, acetylation, methylation, and carboxylation.

34. The method of claim 27, wherein said cell is selected from the group consisting of microorganism, animal cell, and plant cell.

35. The method of claim 34, wherein said microorganism is a yeast or a bacteria.

36. The method of claim 27, wherein said culture preparation is a culture medium, a culture broth, a fermentation medium, or a fermentation broth.

37. The method of claim 36, wherein said fermentation medium is an alcoholic or a lactic fermentation medium.

38. A device for measuring a product as an analysis of the metabolism of a cell in a culture medium comprising;
a first reactor comprising a first oxidation or reduction reaction mixture containing an first enzyme and a cofactor, said first enzyme being adapted to transform the product to be measured causing reduction or oxidation of the cofactor;
a second reactor containing a second oxidation or reduction reaction mixture containing a second enzyme, said second enzyme being adapted to transform further the product transformed in the first reactor causing reduction or oxidation of the cofactor;
a detector for determination of the cofactor reduced or oxidized in the first and/or second reactor.

39. The device of claim 38, wherein the first enzyme is diacetyl reductase.

40. The device of claim 38, wherein the second enzyme is butanediol dehydrogenase.

41. The device of claim 38, wherein the cofactor is selected from the group consisting of pyridine-linked dehydrogenase, flavin-linked dehydrogenase, iron-sulfur protein, a cytochrome, ubiquinone, NAD(H) and NADP(H).

42. The device of claim 41, wherein the cofactor is NAD(H) or NADP(H).

43. The device of claim 38, wherein the second oxidation or reduction reaction mixture further comprises the cofactor of the first reactor.

44. The device of claim 38, wherein the detector determine the concentration of the reduced or oxidized cofactor by measuring light absorbance or electric conductivity.

45. The device of claim 38, comprising a further second reactor to be used as a pre-reactor for eliminating the product transformed that may be present in the sample prior to being transformed in the first reactor.
